(19) United States
(12) Patent Application Publication
(10) Pub. No.: US 2016/0235353 A1
(43) Pub. Date: Aug. 18, 2016

(54) SYSTEM AND METHOD FOR DETECTING INFANT SWALLOWING

(71) Applicant: MOMSENSE LTD., Ramat-Gan (IL)

(72) Inventors: Izhak NAKAR, Tel-Aviv (IL); Eduard RUDYK, Haifa (IL); Osnat EMANUEL, Herzlia (IL)

(73) Assignee: Momsense Ltd., Ramat-Gan (IL)

(21) Appl. No.: 15/022,596

(22) PCT Filed: Sep. 16, 2014
(86) PCT No.: PCT/IL2014/050823

§ 371 Date: Mar. 17, 2016

Related U.S. Application Data
(60) Provisional application No. 61/880,936, filed on Sep. 22, 2013.

(51) Int. Cl. A61B 5/00; A61B 7/04; A61B 7/00

(52) U.S. Cl. CPC A61B 5/4205; A61B 7/008; A61B 5/7282; A61B 5/7278; A61B 5/4312; A61B 5/7405; A61B 7/003; A61B 5/725; A61B 7/04; A61B 2503/04; A61B 2562/0204

(57) ABSTRACT

According to an aspect of some embodiments of the present invention there is provided a method of detecting at least one infant swallowing event comprising: obtaining at least one signal segment of an audio recording of an infant during a breastfeeding session; automatically detecting a first, a second and a third peak region in the at least one signal segment; automatically calculating a first duration from the end of the first region to the start of the second region, and a second duration from the end of the second region to the start of the third region; automatically determining an infant swallowing event if the first duration falls within a first signature time range, and if the second duration falls within a second signature time range; and outputting a signal indicative of the infant swallowing event to a user so that the user is aware of the infant swallowing event.

### FIELD AND BACKGROUND OF THE PRESENT INVENTION

The present invention, in some embodiments thereof, relates to a system and/or a method for detecting swallowing and, more particularly, but not exclusively, to a system and/or a method for detecting infant swallowing during breastfeeding.

Exclusive breastfeeding is recommended by many professional organizations, at least for the first 4 months of a baby's life. Breastfeeding has many benefits, both to the infant and the mother. Some examples include; easy digestion, increased immunological protection, parent-child bonding, loss cost, and convenience.

Inadequate breastfeeding and consequently inadequate milk intake may lead to complications for the infant, for example, breast feeding jaundice, poor weight gain and dehydration.

Gauging the amount of milk consumed by the infant may be difficult. Riordan J et al. “Indicators of effective breastfeeding and estimates of breast milk intake.” J Hum Lact. 2005 November; 21(4):406-12, tested indicators of effective breastfeeding to identify those that provide reliable estimates of human milk intake. The authors found that after 96 hours of life, “audible swallowing alone estimated human milk intake”.

Gakhar et al., in U.S. Patent Application Publication No. 2008/0264180 describe “An audio sensor and receiver detect, discriminate and count the number of liquid swallows to determine the volume of fluid ingested.” The system may be used by nursing mothers to help determine the volume of milk ingested by the infant.

### SUMMARY OF THE PRESENT INVENTION

An aspect of some embodiments of the present invention relates to systems and/or methods for automatically detecting one or more infant swallowing events during a breastfeeding session. In exemplary embodiments, a signal segment is analyzed for durations between three distinct peak regions for signature time ranges in order to automatically detect the infant swallowing event.

According to an aspect of some embodiments of the present invention there is provided a computerized method of detecting at least one infant swallowing event, the method comprising:

obtaining at least one signal segment of an audio recording of an infant during a breastfeeding session;

automatically detecting a first, a second and a third peak region in the at least one signal segment;

automatically calculating a first duration from the end of the first region to the start of the second region, and a second duration from the end of the second region to the start of the third region;

automatically determining an infant swallowing event if the first duration falls within a first signature time range, and if the second duration falls within a second signature time range; and

outputting a signal indicative of the infant swallowing event to a user so that the user is aware of the infant swallowing event.

According to some embodiments of the invention, the first signature time range comprises about 50-300 ms and the second signature time range comprises about 300-1000 ms.

According to some embodiments of the invention, the computerized method further comprises providing initialization data associated with the infant to estimate an amount of milk per infant swallowing event, and estimating a total amount of milk swallowed per feeding session according the estimated milk per infant swallowing event and the number of detected infant swallowing events.

According to some embodiments of the invention, outputting comprises outputting an augmented filtered signal of the detected infant swallowing event so that the user hears infant sounds associated with the detected swallowing event and does not hear sounds not associated with the detected swallowing event.

According to some embodiments of the invention, the audio recording is recorded from an infant with swallowing sounds with a volume too low for the mother to discern.

According to some embodiments of the invention, the first duration corresponds to a pharyngeal stage of the infant swallowing event.

According to some embodiments of the invention, the second duration corresponds to an esophageal stage of the infant swallowing event.

According to some embodiments of the invention, automatically detecting comprises automatically estimating a probability of the infant swallowing event. Optionally, the signal segment is scaled so that the signal segment is used to estimate the probability.

According to some embodiments of the invention, the computerized method further comprises detecting respiration sound patterns, associating the respiration sound patterns with the detected infant swallowing event, and increasing or decreasing the probability of the detected infant swallowing event according to the associations of the respiration patterns.

According to some embodiments of the invention, the computerized method further comprises monitoring the quality of the signal of the at least one signal segment and outputting a signal indicative of the quality so that a user may correct the signal quality.

According to an aspect of some embodiments of the present invention there is provided a system for detecting at least one infant swallowing event during a breastfeeding session, the system comprising:

a sensor for detecting audio signals associated with at least one infant swallowing event, the sensor outputting at least one audio signal segment;

a processor for analyzing the at least one audio signal segment for an infant swallow pattern indicative of the at least one infant swallowing event, the processor comprising:

a peak detection module for detecting a first, a second and a third peak regions in the at least one signal segment;

a time module for calculating a first duration from the end of the first peak region to the start of the second peak region and a second duration from the end of the second peak region to the start of the third peak region;

a swallow detecting module for determining if the first duration falls within a first signature time range, if the second duration falls within a second signature time range, and determining if the swallowing event occurred;

an output module for generating a signal indicative of the swallowing event; and

an output unit for generating an output of one or both of a sound and a picture in response to the signal indicative of the swallowing event.

According to some embodiments of the invention, the processor resides in a smartphone.

According to some embodiments of the invention, the output unit comprises an audio headset and the sensor is attached to the headset.

According to some embodiments of the invention, the system further comprises a wireless transmitter in electrical communication with the sensor and a wireless receiver in electrical communication with the processor.

According to some embodiments of the invention, the system further comprises a high pass filter to filter the at least one audio signal segment above 2.5 kHz to reduce or remove signal components due to one or both of respiration and voice.

According to some embodiments of the invention, the system further comprises one or both of a Gaussian filter and an exponential smoothing filter to filter the at least one audio signal segment so that multiple peaks will generate a relatively higher response.

According to some embodiments of the invention, the system further comprises a memory in electrical communication with the processor, the memory storing data of current and previous infant feeding sessions.

According to some embodiments of the invention, the system further comprises a user input module for the user to control the processor.

According to an aspect of some embodiments of the present invention there is provided a stethoscope for listening to infant swallowing sounds comprising:

a contact element sized for positioning against the skin of an infant's head or neck;

a sensor for generating at least one electrical audio signal segment in response to received sound vibrations from the infant, the sensor is in communication with the contact element so that sound is transmitted from the infant through the contact element to the sensor;

an electrical transceiver for providing electrical communication external of the stethoscope, for transmitting the at least one electrical audio signal segment, and for receiving at least one signal indicative of an infant swallowing event; and

at least one speaker sized for positioning in proximity of or inside an ear canal of a user, the speakers being in electrical communication with the electrical transceiver so that the at least one signal indicative of the infant swallowing event is transmitted by the speakers to the ears of the user.

According to some embodiments of the invention, the stethoscope further comprises a smartphone in electrical connection with the electrical transceiver, the smartphone comprising a signal processing unit for processing the at least one electrical audio signal segment and generating the at least one signal indicative of an infant swallowing event.

According to some embodiments of the invention, the signal processing unit comprises:

a peak detection module for detecting a first, a second and a third peak regions in the at least one audio signal segment;

a time module for calculating a first duration from the end of the first peak region to the start of the second peak region and a second duration from the end of the second peak region to the start of the third peak region;

a swallow detecting module for determining if the first duration falls within a first signature time range, and if the second duration falls within a second signature time range; and

an output module for generating the at least one signal indicative of the infant swallow.

According to some embodiments of the invention, the speakers are detachable from the stethoscope.

According to some embodiments of the invention, the surface area of the contact element is about 0.5-1.0 square centimeters.

According to some embodiments of the invention, a headset of the stethoscope is made of flexible cables.

According to some embodiments of the invention, the electrical transceiver is an integrated Jack Plug for connecting to a mobile device.

Unless otherwise defined, all technical and/or scientific terms used herein have the same meaning as commonly understood by one of ordinary skill in the art to which the present invention pertains. Although methods and materials similar or equivalent to those described herein can be used in the practice or testing of embodiments of the present invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the present invention can involve performing or completing selected tasks manually, automatically, or a combination thereof. Moreover, according to actual instrumentation and equipment of embodiments of the method and/or system of the present invention, several selected tasks could be implemented by hardware, by software or by firmware or by a combination thereof using an operating system.

For example, hardware for performing selected tasks according to embodiments of the present invention could be implemented as a chip or a circuit. As software, selected tasks according to embodiments of the present invention could be implemented as a plurality of software instructions being executed by a computer using any suitable operating system. In an exemplary embodiment of the present invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the present invention are herein described, by way of example only, with reference to the accompanying drawings. With specific reference now to the drawings in detail, it is stressed that the particulars shown are by way of example and for purposes of illustrative discussion of embodiments of the present invention. In this regard, the description taken with the drawings makes apparent to those skilled in the art how embodiments of the present invention may be practiced.

In the drawings:

FIG. 1 is a flowchart of a computerized method of detecting a swallowing event from an audio recording, in accordance with exemplary embodiments of the present invention;

FIG. 2 is an exemplary signal segment indicative of the detected swallowing event processed using the method of FIG. 1, in accordance with exemplary embodiments of the present invention;

FIG. 3 is a block diagram of an exemplary system for executing the method of FIG. 1 to detect the swallowing event, in accordance with exemplary embodiments of the present invention;

FIG. 4 is a schematic illustration of a stethoscope for detecting the swallowing event, in accordance with exemplary embodiments of the present invention;

FIG. 5 is a screen shot of a smartphone running software for detecting the swallowing event of FIG. 1, in accordance with exemplary embodiments of the present invention;

FIG. 6 is a flowchart of a method of nursing a baby by detecting swallowing events using the method of FIG. 1, in accordance with exemplary embodiments of the present invention; and

FIG. 7 is a schematic illustration of another embodiment of the stethoscope for detecting the swallowing event, in accordance with exemplary embodiment of the present invention.

### DESCRIPTION OF SPECIFIC EMBODIMENTS OF THE PRESENT INVENTION

An aspect of some embodiments of the present invention relates to a computerized method for detecting one or more infant swallowing events by an analysis of a unique duration pattern which is recorded using sensors placed to record the swallowing sounds from the baby's skin. In exemplary embodiments, the swallowing event is detected during a breastfeeding session. In exemplary embodiments, a signal segment is analyzed for the presence of signature durations between distinct peak regions.

As used herein, the phrase infant swallowing event means an infant taking a sufficient amount of milk in his/her mouth so that the swallowing reflex is triggered.

Inventors discovered that the durations between the peak regions commonly fall within signature time ranges for different infants. Amplitude and/or duration of peaks may vary from infant to infant and/or from feeding to feeding. However, unexpectedly, the durations between the peak regions remain constant within the signature time ranges for different infants and/or for the same infant during different times.

In exemplary embodiments, a first duration is detected between the end of a first peak region and the start of a second peak region.

In exemplary embodiments, a second duration is detected between the end of the second peak region and the start of a third peak region.

In exemplary embodiments, a first signature time range for the first duration is about 50-300 milliseconds (ms), or about 50-250 ms.

In exemplary embodiments, a second signature time range for the second duration is about 0.35-1 second, or about 0.3-1 second, or about 0.5-1 second.

Optionally, the durations (i.e., length) of the peak regions are variable.

Optionally, the amplitudes of the peak regions are variable.

Optionally, the signal segment is analyzed for the presence of a swallow pattern. Optionally, the swallow pattern comprises a pharyngeal phase and an esophageal phase. Optionally, the swallow pattern is indicative of the swallow event.

Optionally, a raw audio recording of the infant during the breastfeeding session is processed so that the processed signal contains only sounds associated with the detected swallowing event. Optionally, the processed signal does not contain other sounds, for example, voices, infant respiration, background noise, and/or swallowing sounds having a low probability score. Optionally, the processed signal is amplified to be heard by a user, for example, through earphones.

Optionally, infant respiration patterns are detected in the signal. Optionally, inspiration and/or expiration are identified. Optionally, the infant respiration pattern is compared with the detected swallowing pattern to increase or decrease the probability of the detected swallowing event.

An aspect of some embodiments of the present invention relates to a stethoscope system for detecting infant swallowing events. In exemplary embodiments, the stethoscope system comprises a headset having integrated earphone(s), an integrated Jack Plug for connecting to a mobile device, an integrated stethoscope microphone and a signal processing module for running on the mobile device, for example a Smartphone or Tablet application, to convert a raw audio signal recorded using the stethoscope microphone into a processed signal indicative of one or more infant swallowing events.

In use, the user holds a contact element of the stethoscope microphone against the infant's skin (e.g., head, jaw, neck) during the feeding session, while listening to the processed signal and/or other related signals from the headset. Advantageously, the stethoscope system may be used with only one hand, leaving a second hand free.

In exemplary embodiments, the signal processing unit is external to the stethoscope housing, for example, stored on a memory of a smartphone. Optionally, the stethoscope comprises a transceiver for communicating with the smartphone, such as a wireless transceiver and/or a cable. Alternatively, the signal processing unit is integral with a housing of the stethoscope.

As used herein, the term transceiver means one or more components for transmitting and/or receiving data.

Optionally, the stethoscope microphone has a contact element sized and shaped for contacting the skin of the infant to transmit sounds from the infant to the stethoscope, for example, the infant's head, jaw and/or neck. The surface area of the contact element is, for example, about 0.5-1.0 square centimeters (cm²), or about 0.7-4 cm², or other smaller, intermediate or larger ranges.

The present invention, in some embodiments thereof, relates to a system and/or a method for detecting swallowing and, more particularly, but not exclusively, to a system and/or a method for detecting infant swallowing during breastfeeding.

Before explaining at least one embodiment of the present invention in detail, it is to be understood that the present invention is not necessarily limited in its application to the details of construction and the arrangement of the components and/or methods set forth in the following description and/or illustrated in the drawings and/or the Examples. The present invention is capable of other embodiments or of being practiced or carried out in various ways.

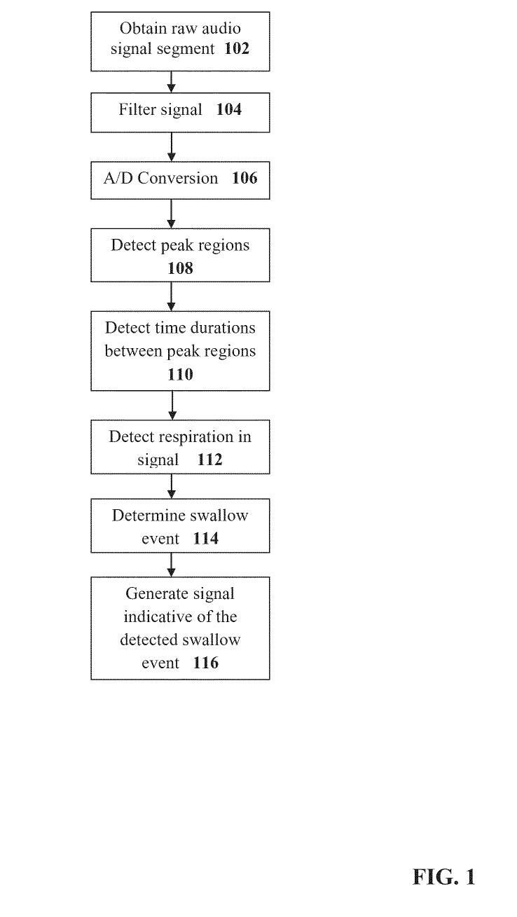

Referring now to the drawings, FIG. 1 illustrates a flowchart of a computerized method of automatically detecting a swallowing event from an audio recording, in accordance with exemplary embodiments of the present invention. In exemplary embodiments, the swallowing event is detected by recognition of a signature pattern discovered by Inventors. The signature pattern is comprised of a first duration between first and second peak regions, and a second duration between second and third peak regions. Advantageously, the method may detect swallowing events that the mother is unable to hear (e.g., due to background noise and/or low volume) and/or may confirm to the mother that what she heard was in fact a swallowing event.

At 102, a recording of the infant during the feeding session is obtained. Optionally, the recording is an unprocessed raw signal. Optionally, the recording is an audio recording. Optionally, the recording is an analogue signal. Optionally, the recording is a time domain signal.

Optionally, the recording is divided into signal segments. The segments may be contiguous and/or overlapping. Alternatively, the recording is obtained in a continuous manner. Optionally, a sliding window is used to analyze the signal.

In exemplary embodiments, the signal segment is long enough to contain a representation of the swallow pattern, the swallow pattern comprising of the pharyngeal phase and the esophageal phase. The length of the signal segment is, for example, about 550-1250 milliseconds (ms), or about 600-2000 ms, or about 1-3 seconds, or other intermediate or larger lengths. Alternatively, the signal segment is not long enough to contain the full representation of a swallow pattern. Optionally, the short signal segments are analyzed for the presence of peaks and/or durations indicative of the swallow pattern, the short signal segments being synchronized to detect the complete swallow pattern.

At 104, the raw signal and/or signal segments are filtered. Optionally, the raw signal is filtered by an analogue filter. Optionally, the filter is selected to remove parts of the signal that are unrelated to the swallowing pattern, for example, very high frequencies (e.g., above the Nyquist frequency) and/or very low signals (e.g., below the human hearing range).

Alternatively or additionally, the signal is filtered by a high pass filter having a cutoff of about 2.5 kilohertz (kHz). Optionally, the high pass filter is a digital filter, the signal being filtered by the digital filter after conversion into digital format (e.g., box 106). Other examples of possible cutoffs for the high pass digital filter include: about 2 kHz (may still filter 2^{nd} harmonics of respiration), about 3 kHz (may also filter the 3^{rd} harmonics of the signal).

Optionally, the filter is selected to remove low frequency signal components, for example, heart beats, respiratory signals, and/or voice signals.

Alternatively or additionally, one or more filters are applied at boxes below, optionally after the signal has been digitized (e.g., by box 106), for example, the 2.5 kHz high pass filter is applied at box 108, a digital band-pass filter is applied at box 112 (may enhance respiratory signals between about 700 Hz-900 Hz).

Advantageously, the cutoff frequency of 2.5 kHz may filter out energy changes due to respiration, (harmonics may be filtered out), and reduce the effect of voice in the signal, for the voice frequency band of about 300 Hertz (Hz)-3 kHz.

Alternatively or additionally, the signal is filtered by a Gaussian filter and/or an exponential smoothing filter. Optionally, the Gaussian and/or exponential filters are selected so that the signal segment with multiples peaks will generate a relatively higher response in the filtered signal. Advantageously, the higher response may allow for easier detection of peak regions, as will be described in more detail below with reference to box 108.

Alternatively or additionally, the signal is filtered to remove respiration signals. Advantageously, the 2.5 kHz digital high pass filter may remove the first harmonics of respiratory signals (about 800 Hz, or about 900 Hz) and/or the second harmonics (about 1500-1800 Hz, or about 1900 Hz).

Alternatively or additionally, the signal is scaled. Advantageously, the scaled signal may be interpreted as a probability function, for example, in probabilistic decision schemes such as a Hidden Markov Model, for example, as described with reference to box 114.

At 106, the analogue filtered signal segment is converted into digital format.

Alternatively, the order of analogue to digital (A/D) conversion and filtration is reversed, so that the raw signal is first converted into digital format and then filtered, for example, box 106 is performed before box 104, for example, the signal is A/D converted and then filtered by a high pass digital filter having a cutoff of about 2.5 kilohertz (kHz).

Alternatively, the raw analogue signal is first filtered by an analogue filter (e.g., 104), then A/D converted (e.g., 106), and then filtered by a digital filter (e.g., 104 again).

At 108, one or more peak regions are detected in the signal segment.

Each peak region comprises of at least one individual peak. The peak region may comprise of a cluster of peaks that are close together.

Optionally, the noise level is estimated, for example, by using exponentially weighted moving average with peaks filtering.

Optionally, the peaks are detected when the signal to noise ratio (SNR) crosses a predefined threshold.

Alternatively or additionally, a peak region is detected by a stretch of background noise that precedes and follows the peak region. The background noise is detected by a signal to noise ratio less than the threshold selected for detecting the peak region. The background noise level may differ and/or vary before, after and/or between peak regions, for example, the noise level during swallowing may be lower than the noise level before and/or after the swallowing.

A peak region is detected as having a duration of no more than about 5 milliseconds (ms), or about 10 ms, or about 30 ms, or other smaller, intermediate or larger values. Optionally, the peak is detected as a local maximum above the threshold SNR, using a window of size about 5 ms, or about 10 ms, or about 30 ms.

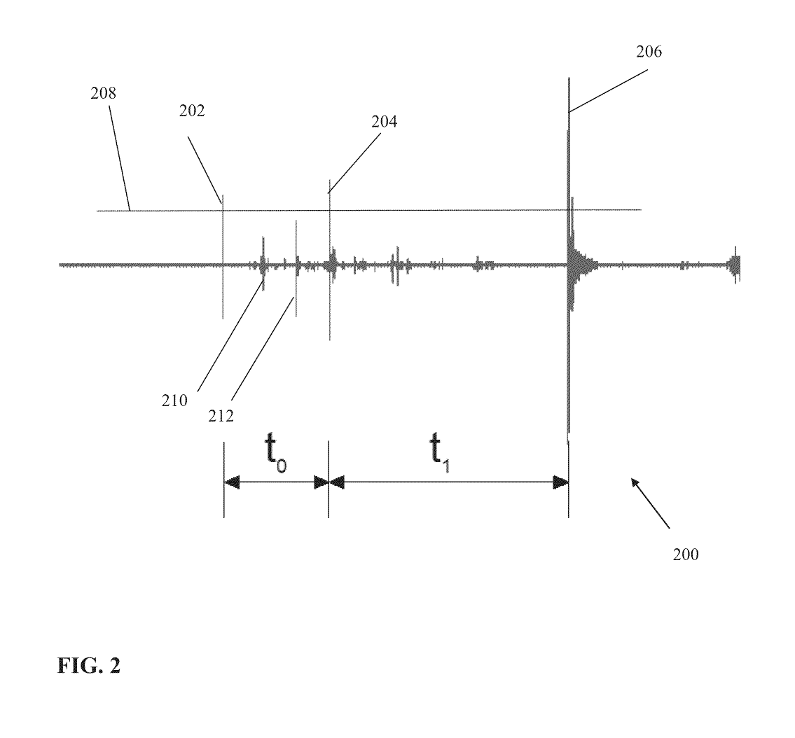

Multiple peak regions are detected as being spaced apart in time, as having a period of time between one region and another region that does not include peaks that fall into either region. Smaller peaks falling within the space between the regions are not classified as a region. Multiple peak regions are detected as being distinct, and do not overlap with one another. Additional details of exemplary spacing between the regions will be discussed with reference to t₀ and t₁ of FIG. 2.

A peak region may be detected by one or more increases in the amplitude of the signal (i.e., one or more peaks) relative to background noise levels, for example, by a processor analyzing the signals. The value of the highest peak and/or central peak and/or the average of the peak values or other measurements in the peak region, may be divided by the energy of the background signal. A peak may be detected if the signal to noise (i.e., background signal) ratio is over a preselected threshold. The threshold may be selected, for example, according to experimental data collected by the manufacturer, automatically detected by the system depending on the ambient noise level, preset by the manufacturer and/or user.

In exemplary embodiments, at least 3 peak regions are detected. The 3 detected peak regions are further analyzed for signature patterns that suggest that the swallowing event occurred.

Without being bound to theory, the first peak region and the second peak region correspond to the first stage of swallowing. A third distinctive peak region corresponds to the relaxation stage, which occurs at the end of the swallowing pattern. At 110, the duration between the peak regions is determined. The duration is calculated as the time from the end of one peak region to the start of the next peak region.

The presence of a signature duration pattern is detected by matching a reference pattern. The reference pattern comprises a first signature duration between a first and a second peak, and a second signature duration between the second and a third peak.

The reference signature time pattern comprises the first signature duration in the range of about 50-300 ms, and the second signature duration in the range of about 350 ms-1 second.

Optionally, at 112, respiration patterns are detected in the signal segment. Optionally, inspiration and/or expiration are detected, for example, by the duration of expiration being longer than the duration of inspiration.

Optionally, respiration patterns are detected in the frequency domain of the signal segment. Alternatively or additionally, respiration patterns are detected in the time domain of the signal segment.

Optionally, respiration patterns are detected by the presence of relatively high energy amplitude (e.g., above a predetermined signal to noise threshold) at about 800 Hz and/or at 1.7 kHz. Optionally, a band-pass filter is applied between about 700 Hz-900 Hz.

Optionally, the respiration pattern relative to the signal time pattern and/or relative to the detected peak regions is detected.

Optionally, the respiration pattern is detected by an increase in amplitude of the signal in the frequency range of about 800-900 Hz.

Optionally, the increase in signal amplitude corresponding to inspiration and/or expiration is correlated with the detected swallowing pattern. Optionally, the signal amplitude is detected before and/or after the detected swallowing pattern.

At 114, the presence of a swallowing event is determined.

Optionally, the presence of a swallowing event is determined as a binary event: the swallowing event occurred, or the swallowing event did not occur.

Optionally, the swallowing is determined to have occurred if the signature time pattered (e.g., as described with reference to box 110) has been detected in the signal segment.

Alternatively or additionally, the presence of a swallowing event is determined as a probability that the swallowing event occurred. Optionally, the probability is converted into the swallowing event or no swallowing event, by using a preselected probability threshold value. A probability above the threshold is converted into the swallowing event, or a probability below the threshold is converted into the no swallowing event.

Optionally, the scaled signal (e.g., as described in box 104) is interpreted as a probability function and used in a probabilistic decision schemed, for example, the Hidden Markov Model. Event detection may be viewed as a transition of states, for example, the states of: first peak, second peak, third peak. State transitions may be detected by a suitable model, for example, a Markov model. The input may be the detected peaks. The output may be the probability of the event. The Markov model may be used to implement boxes 110 and/or 114.

Optionally, the respiration pattern determined in box 112 confirms the swallowing event and/or increases the probability of the swallowing event, if the swallowing pattern is preceded by the inspiration signal pattern and/or is followed by the expiration signal pattern. Alternatively, the respiration pattern invalidates the swallowing event and/or decreases the probability of the swallowing event, if the inspiration and/or expiration occur at the same time as the swallowing event.

At 116, a signal indicative of the detected swallowing event is generated. Alternatively, the signal is indicative that no swallowing event has been detected. Alternatively, the signal is indicative of the probability that the swallowing event occurred. Alternatively, the signal is indicative of low signal quality.

Optionally, the signal is a filtered version of the original audio recording, with sounds that do not correspond to the detected swallowing event having been removed. Alternatively or additionally, peak regions that do not correspond to the detected swallow have been removed. Alternatively or additionally, respiration patterns have been removed.

Reference is now made to FIG. 2, which is an exemplary signal segment 200 indicative of a swallowing event, in accordance with exemplary embodiments of the present invention. The signal has been processed and/or analyzed using the method described with reference to FIG. 1. For clarity, respiration related signals are not shown.

Signal segment 200 is shown in the time domain.

Signal segment 200 is shown after having been processed by a high pass filter with a cutoff frequency of 2.5 kHz and normalized to noise level, for example, as described with reference to box 108 of FIG. 1.

Three peak regions 202, 204 and 206 have been detected, for example, as described with reference to box 108 of FIG. 2. Three peak regions correspond to the number of peak regions required to determine the swallow pattern.

Optionally, the peaks are detected based on the selection of a suitable threshold 208. Peaks with amplitudes above threshold 208 are identified. If the threshold is set too low, other invalid peaks may be detected, for example, 210 and/or 212. Peak regions 210 and 212 may be eliminated from consideration as being valid peaks when the time signature is determined, as described below.

t₀ denotes the first signature duration, and t₁ represents the second signature duration, for example, as described with reference to box 110. Peak regions 210 and 212 are determined as being invalid as the durations measured to and/or from peak regions 210 and/or 212 to other peak regions do not fit the signature durations.

As the signal segment contains three peak regions 202 204 and 206 with durations t0 and t1 that satisfy the signature durations, the signal segment may be indicative of the swallowing event.

Inventors discovered that signal 200 corresponds to the swallowing cycle leading to the swallowing event. Without being bound to theory, the first part of the signal, represented by t0, corresponds to the pharyngeal phase of the swallowing reflex, such as closure of the nasopharynx and the larynx. The second part may correlate with the end of the pharyngeal stage, and/or the beginning of the esophageal stage. During these stages the milk is propelled through the esophagus to the stomach. The end of the second part may correlate with the final stage of the swallowing-relaxation stage. The final peak may correlate with the relaxation stage, as the pharynx and larynx move back to their resting location, ready for another swallowing cycle.

Inventors discovered that the duration of a peak region may vary between infants, and between the same infant. Inventors discovered that both durations between the three peak regions are constant within the first and second signature time ranges, for different infants and/or for the same infant at different times.

Inventors discovered that the first part may correlate with several peaks, such as peaks 202, 210, 212 and/or 204. Inventors discovered that there are 2 peak regions with relatively higher amplitudes than the other peaks (e.g., above threshold 208) that have the distinct time signature between them that falls within a signature time range, represented by t0. The time between first peak 202 and second peak 204, also shown as t0, ranges from about 50-300 ms.

Inventors discovered that the second part may correlate with no peaks (e.g., all below threshold 208), expect for the beginning and end. The end of the second part may correlate with one or a few closely spaced peaks, for example peak region 206. Peak 206 may correlate with the end of the swallowing cycle. The duration of the second stage, between second peak 204 and third peak 206, also shown as t1, ranges from about 350 ms-1 second.

Inventors discovered that the swallowing cycle may be preceded by inspiration and/or may be followed by expiration. The inspiration and/or expiration may be detected by an increase in amplitude in the frequency band, such as 800 Hz-900 Hz before and/or after the swallowing pattern.

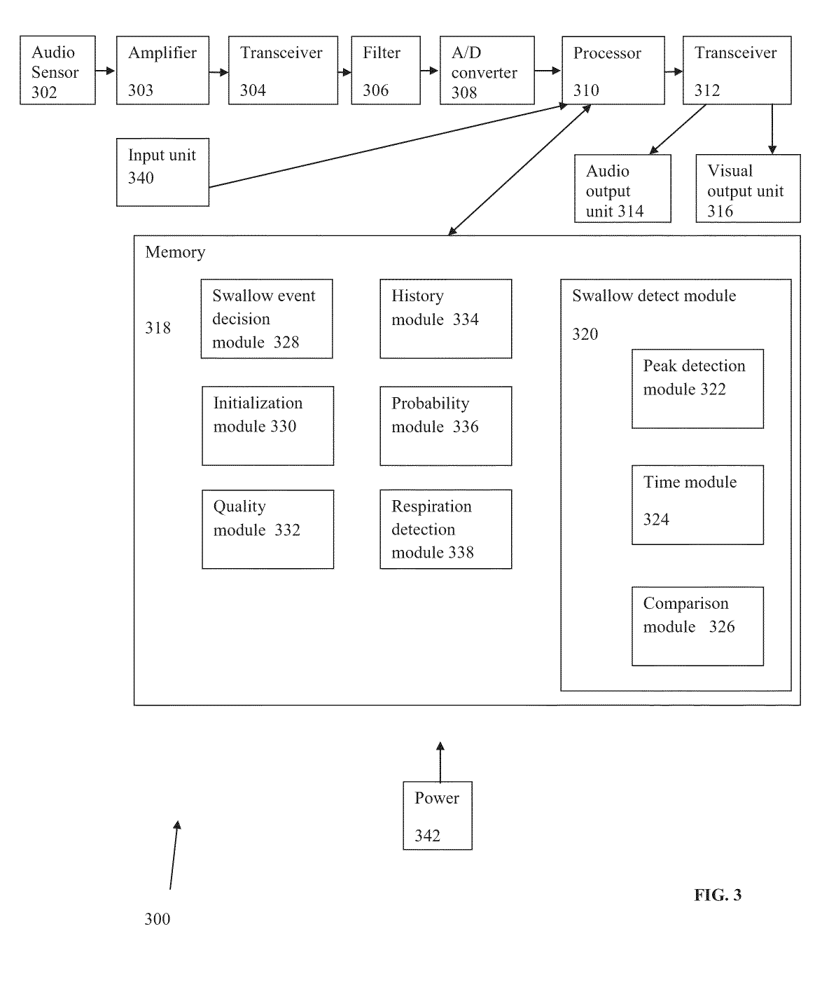

Reference is now made to FIG. 3, which is a block diagram of a swallow detection system 300 for detecting an infant swallowing event, in accordance with embodiments of the present invention. In exemplary embodiments, system 300 executes the automated method described with reference to FIG. 1.

Optionally, an audio sensor 302 converts sound vibrations associated with infant swallowing sounds into an analogue electrical signal. Alternatively, sensor 302 is a digital sensor generating a digital signal. Examples of suitable sensors 302, include; a contact microphone, a non-contact microphone, an accelerometer, a vibration sensor, or other suitable sensors, for example, a unidirectional microphone model CM1045RFH-35BL-C56F1K-NF-LF available from MWM Acoustics®.

Optionally, sensor 302 is sized for positioning against the infant, for example, the neck of the feeding infant. Alternatively, sensor 302 is positioned in proximity to the infant without direct contact.

Optionally, an amplifier 303 amplifies the analogue signal.

Optionally, the amplified analogue signal is filtered by one or more filters 306, for example, the high pass filter, the Gaussian filter, and/or the exponential smoothing filter as described with reference to box 104 of FIG. 1. The filters may be analogue and/or digital. The filters may be applied before and/or after A/D conversion.

Optionally, the filtered analogue signal is converted into digital format by an analogue to digital (A/D) converter 308.

Alternatively, the order of components 303, 306 and/or 308 is interchanged. For example, the analogue recording may first be converted into digital format by A/D converter 308, then amplified by amplifier 303 and then filtered by filter 306. In such a case, amplifier 303 is a digital amplifier, and filter 306 is a digital filter that may be implemented as circuitry or as a software module. In another example, the analogue recording is first amplified by amplifier 303, then converted by A/D converter 308, and then filtered by filter 306.

The digital signal is analyzed and/or processed by a processor 310.

A memory 318 in electrical communication with processor 310, stores thereon several modules of instructional code for execution by processor 310.

An optional initialization module 330 provides initialization data and/or instructions for system 300 before the infant feeding session has started. Optionally, the initialization data comprises of demographic information about the infant, for example, age, weight, sex, geographical location and/or nationality of the infant. The initialization data may be entered before the first use of system 300, saved for subsequent sessions, and/or updated with changes.

The initialization demographic data may be used to estimate the volume of milk per infant swallowing event. The estimates may be provided by a look-up table based on experimentally collected data, and/or by a mathematical model.

Inventors discovered that the relationship between swallowing events and consumed volume is non-linear, as the infant consumes more at the beginning of the session and less towards the end of the session. Alternatively, a linear model may be used (the linear model may be simpler to implement). For example, the total volume of milk per feeding session may be estimated, for example, by multiplying the number of swallowing events by the estimated volume of milk per infant swallow. An infant may swallow about 0.3-0.5 (milliliters) mL of milk per detected swallowing event. If about 100-120 swallowing events were detected for the session, the total estimated amount of milk swallowed for the session is about 10-60 mL, or about 35 mL on average.

Optionally, the initialization instructions comprise of system check for errors, for example, the microphone not being attached, low batteries, and/or other types of errors.

A swallow detect module 320 detects the infant swallowing pattern in the signal, as described with reference to FIG. 1. A peak detection module 322 detects the peak regions in the signal, as described with reference to box 108 of FIG. 1. A time module 324 detects the durations between the detected peak regions, as described with reference to box 110 of FIG. 1. A comparison module 326 compares the pattern of detected peak regions and the detected durations between the peak regions to the signature duration pattern, as described with reference to box 110 of FIG. 1.

An optional respiration detection module 338 detects respiration patterns in the signal, as described with reference to box 112 of FIG. 1.

An optional probability module 336 estimates the probability of the infant swallowing pattern being indicative of the infant swallowing event, as described with reference to box 114 of FIG. 1.

A swallowing event decision module 328 analyzes the output of swallow detect module 320, probability module 336 and/or respiration detect module 338, and provides appropriate output. The output may be a signal indicative of the swallowing event, a signal indicative that the swallowing event did not occur, and/or a signal indicative of the probability that the swallowing event occurred.

Some examples of audio output include: augmented sounds associated with the swallowing event (lower volume for other sounds), only sounds associated with the swallowing event, a beep after the swallowing event, a verbal message after the swallowing event, for example “Infant swallow”.

Some examples of video output include: an image of a baby with exaggerated swallowing corresponding to the infant swallowing events, a light after the swallowing event, a number indicating the cumulative number of swallowing events of the session, a percentage probability that the swallowing event occurred, the estimated cumulative volume of milk consumed during the session, a text message following the event, for example “Infant swallow”, a wave-like animation.

An optional quality module 332 monitors quality before and/or during the feeding session. For example, the quality of the audio recording, the background noise level, and/or the amplitude of the signal. Optionally, the quality of the audio recording is monitored, for example, by comparing the measured signal to noise ratio against expected or historical signal to noise ratios. If the signal to noise ratio is low, the user may be prompted to check the position of the microphone, and/or move into a quieter environment. Optionally, the quality of the feeding is monitored, for example, by comparing the rate of swallowing events over time against expected or historical rates. The user may be prompted to check the feeding position of the baby.

Optionally, quality module 332 uses raw data to estimate missing signal and/or loud noise events.

An optional history module 334 summarizes one or more parameters associated with the feeding session. Data may be presented in an ongoing manner during the feeding, or as a summary report after the feeding. Optionally, data of previous sessions are saved for future review and/or analysis. Examples of the data parameters include; total estimated volume of consumed milk to date for the current feeding session, and/or comparison of the feeding rate of the current session to past sessions.

One or more of amplifier 303, filter 306, A/D converter 308, memory 318 and/or processor 310 are made from off the shelf components. Alternatively or additionally, the components are made from custom manufactured designs.

An optional input unit 340 is adapted to interface with the user to allow the user to enter data into system 300. Examples of input unit 340 include; a keyboard, a touch screen, a mouse, a touchpad, a voice analyzer and/or a button.

An optional audio output unit 314 is adapted to interface with the user to allow the user to hear sounds outputted by system 300. Examples of output unit 314 include speakers, earphones, and/or headphones.

An optional visual output unit 316 is adapted to interface with the user to allow the user to see images and/or videos outputted by system 300. Examples of output unit 316 include; a video screen, blinking lights, color coded lights, and/or a segment display (i.e., numbers or alphanumeric characters). Alternatively or additionally, output unit 316 is adapted so that the user receives tactile output. Advantageously the system may be used by blind and/or deaf mothers.

Optionally, input unit 340 and visual output unit 316 are combined into a single unit, for example, a touchscreen.

Reference is now made to FIG. 5, which is a screen shot 500 of a smartphone running software for detecting the swallowing event of FIG. 1 and/or comprising processor 310, in accordance with exemplary embodiments of the present invention.

Optionally, a button 502 is pressed on the touchscreen to start and/or stop the processing of the audio recording (shown during processing).

Optionally, the screen provides indications of the swallowing event, for example, by flashing, changing colors and/or displaying a picture.

Optionally, a menu function 504 displays a pull down menu with different options, for example, to view data and/or select options of one or more software modules 330, 332, 334, 336, 338, 328, 320 described herein.

Optionally, a screen view button 506 changes screen views, for example, to a screen showing data of the feeding, for example, total swallowing events to date, and/or total estimated milk consumption. Advantageously, the single large start/stop button on the screen allows the mother to easily start and stop the signal analysis, for example, during position adjustments of the baby.

Optionally, the software module comprises one or more additional functions, for example:

Reminders and/or instructions to the mother, for example, reminder to position the sensor, reminder to log the data, reminder to save the data, reminder to download new versions of the software, reminder to charge the battery, a reminder to change the diaper of the baby, or other reminders.

A report generator function for generating a report of: real time statistics, to-date session statistics, statistics of all previous sessions. For example, tracking the total volume of milk per session over time. The reports may help the mother track progress in feeding the baby.

A graph generator for generating visual representations of the report statistics.

The report and/or graphs may show a comparison of the feeding statistics of the infant to average feeding statistics of infants for similar ages. The comparison may help the mother track if her baby is feeding more or less than expected.

A weight calculator and or estimator, for tracking the weight gain progress of the infant. The weight of the infant may be estimated based on the feeding statistics. Inadequate weight gain or weight loss may be detected early using the estimator. Actual measured weights may be entered and tracked as well.

A Feeding Time Monitor, for tracking the amount of time spent feeding. The Monitor may provide alarms, for example, an alarm to switch the feeding breast.

A breastfeeding troubleshooting guide, that may provide visual and/or textual assistance to help the mother if she is having trouble. For example, how to position the baby, how to latch the baby, how to hold the baby while feeding, signs and/or symptoms of mastitis, signs and/or symptoms of engorgement. A button may be pressed to automatically call a healthcare provider for assistance.

An automatic smartphone configuration based upon detection of the feeding session, for example, blocking all incoming calls, automatically responding with an SMS message “I will get back to you later”, loading previous statistics and/or settings.

A power supply 342 provides power to one or more of the components of system 300. Power supply 342 may be a plug to connect to a wall outlet and/or a battery, optionally rechargeable. Power supply 342 may provide enough power to last a feeding session, optionally multiple feeding sessions.

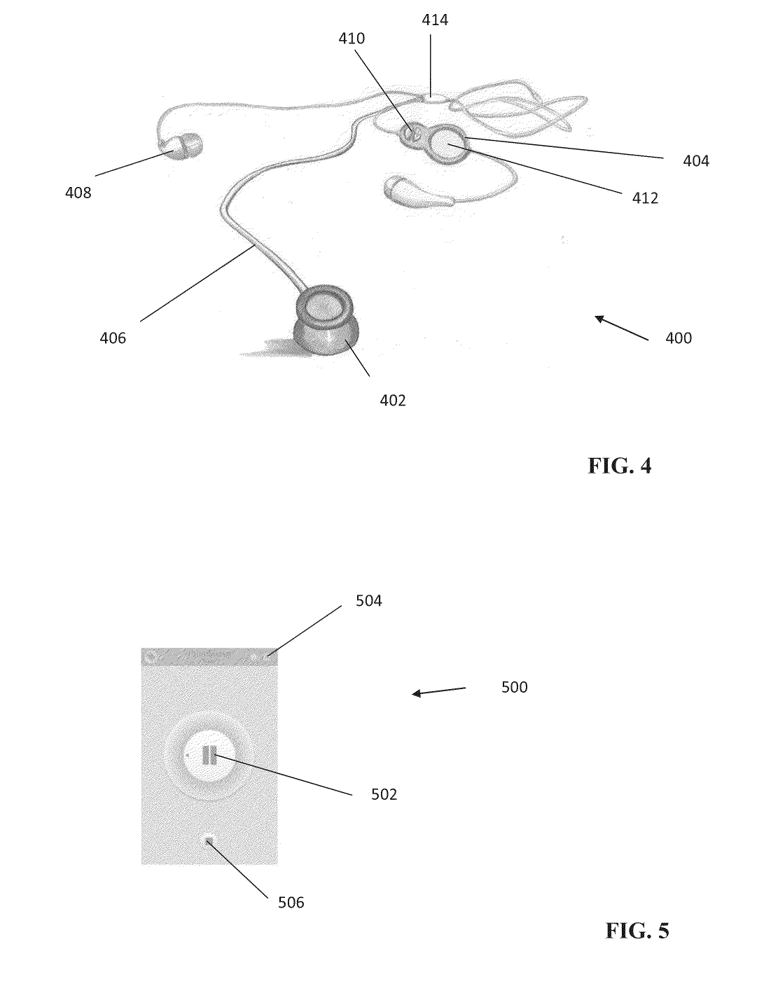

Optionally, all of the components are integrated into a single device, for example, the stethoscope as described with reference to FIG. 4. Alternatively, the components are arranged into two or more separate devices, with communication between devices provided by one or more transceivers 304 and/or 312. Optionally, some components are replaceable and/or detachable. For example, sensor 302 may be taped to the neck of the baby, transmitting signals through an antenna. Filter 306, A/D converter 308, processor 310, memory 318, input unit 340 (e.g., touchscreen), and/or output units 314 (e.g., speaker) and 316 (e.g., screen) may be part of a smartphone having a software program stored thereon. Earphones may alternatively be plugged into the smartphone so that audio output may be heard by the user without disturbing the feeding baby. In another example, sensor 302 transmits (via a wired and/or wireless connection) the signal to an external and/or remote server for processing. The processed signal is then received by transceiver 312 and played by audio unit 314 and/or displayed on output 316 so that the user is provided with data of the feeding session.

Reference is now made to FIG. 4, which is a schematic illustration of a stethoscope 400 for detecting the swallowing event, in accordance with exemplary embodiments of the present invention. Advantageously, stethoscope 400 may be used with only one hand or no hands, may be small, portable and/or easy to use.

In exemplary embodiments, stethoscope 400 transmits sounds associated with a detected fetal swallowing event. Optionally, other sounds are filtered. Alternatively, stethoscope 400 also transmits some other sounds, for example, inspiration and/or expiration. Advantageously, the isolated sounds of the baby swallowing may be reassuring and/or comforting to the mother, while allowing the mother to monitor the milk intake of the baby.

Stethoscope 400 comprises of a contact element for positioning against the skin of the baby to receive vibrations from the baby, for example, a chestpiece 402 used in standard physician stethoscopes, or a smaller version thereof. Chestpiece 402 may comprise a bell and/or a diaphragm. Alternatively, chestpiece 402 is a contact microphone, and/or other circuitry for converting vibrations into electrical signals.

Optionally, chestpiece 402 is arranged for easy holding between two fingers, or for being grabbed between two fingers. Advantageously, chestpiece 402 may be gently positioned against the skin of the infant without application of pressure to disturb the infant.

Optionally, chestpiece 402 is in communication with a signal processing unit so that sounds are transmitted from chestpiece 402 to a signal processing unit. Optionally, a tube 406 is hollow for transmitting vibrations through air. Alternatively, tube 406 comprises wires for transmitting electrical signals.

Optionally, connector 414 serves as a separation point between the cables to the left and/or right speakers and/or microphone. Optionally, the mobile devices comprises a signal processing unit for producing an output signal indicative of and/or associated with the infant swallowing event. An example of a suitable signal processing unit has been described with reference to FIG. 3. An example of a method has been described with reference to FIG. 1. An example of a smartphone has been described with reference to FIG. 5.

Alternatively, stethoscope 400 comprises a wireless communication element adapted to communicate with an external and/or remote processor. For example, a remote server operated by a healthcare provider may be accessed through a wireless internet connection. Advantageously, the device may be used by mothers that do not own a smartphone, but want the application functions provided by the mobile device. Alternatively, the signal processing unit is integrated with stethoscope 400, for example, residing in unit 404.

Headset 408 receives the output signal and generates sound output for the mother to hear. Optionally, headset 408 comprises of flexible cables. Advantageously, the flexible cables may be compactly arranged for portability. Alternatively, headset 408 comprises of rigid tubing, for example, as in stethoscopes used by physicians.

Optionally, one or more buttons are adapted for a user interface to control the signal processing unit on the smartphone. For example, an on/off button, volume buttons, and/or menu navigation buttons.

Optionally, a screen 412 is adapted for visual output of the signal generated by the signal processing unit.

Optionally, components 402, 406, 404, 410 and 408 are integrated into a stethoscope. Optionally, the stethoscope also includes screen 412.

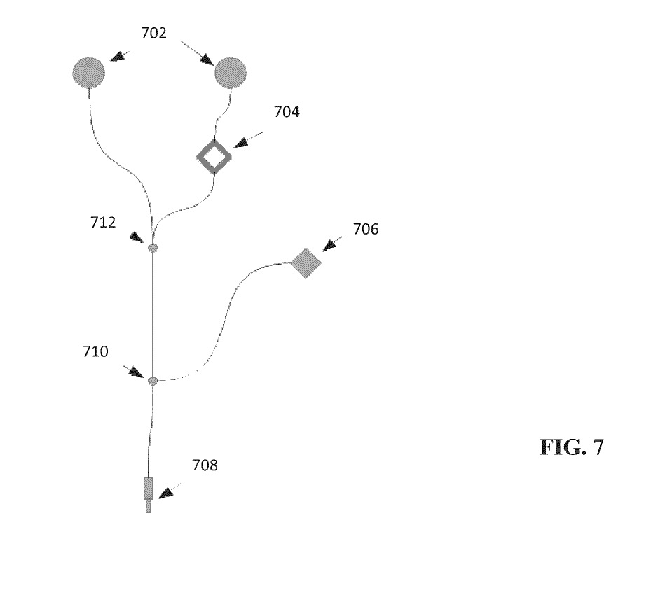

FIG. 7 is a schematic illustration of another embodiment of a stethoscope 700 for listening to infant swallowing, in accordance with exemplary embodiment of the present invention. Stethoscope 700 is made of detachable components. Advantageously, the detachable components may be replaceable (e.g., if broken) and/or upgraded.

Stethoscope 700 comprises speakers 702 sized for insertion into and/or for proximal placement near ear canals of a user. Optionally, a microphone 706 is sized to fit in a base 704, for example, for storage, and removed from base 704 during the feeding session. For example, microphone 706 clicks in and out of base 704, microphone 706 attaches to base 704 with a reusable adhesive, and/or microphone 706 connects to base 704 using a hook-and-loop fastener. A jack-plug 708 is adapted for attachment to a mobile device, for example, a smartphone. Optionally, microphone 706 is detachable from stethoscope 700 at separation point 710. Optionally, the left and/or right speakers 702 are detachable from stethoscope 700 at separation point 712.

Alternatively, one or more components are detachably connected to one another. Optionally, a connector detachably mechanically and electrically communicates between headset 408, tubing 406 and/or unit 404. Advantageously, headset 408 may be detached and plugged in other devices, for example, to hear music and/or for making phone calls.

Optionally, the headset 408 and/or tube 406 are connectable to the mobile device. Tube 406 may be plugged into the input of the mobile device. Headset 408 may be plugged into the output of the mobile device.

Alternatively, an off the shelf electrical stethoscope is programmed with the method of FIG. 1 and/or contains the modules of FIG. 2.

Optionally, stethoscope 400 is adapted to analyze fetal movements. The fetal movements may be heard by placing the stethoscope on the skin of the mother's abdomen over the location of the fetus. Optionally, stethoscope 400 detects fetal swallowing events and/or fetal heart rate. Advantageously, fetal swallowing and/or fetal heart rate may be an indicator of fetal health.

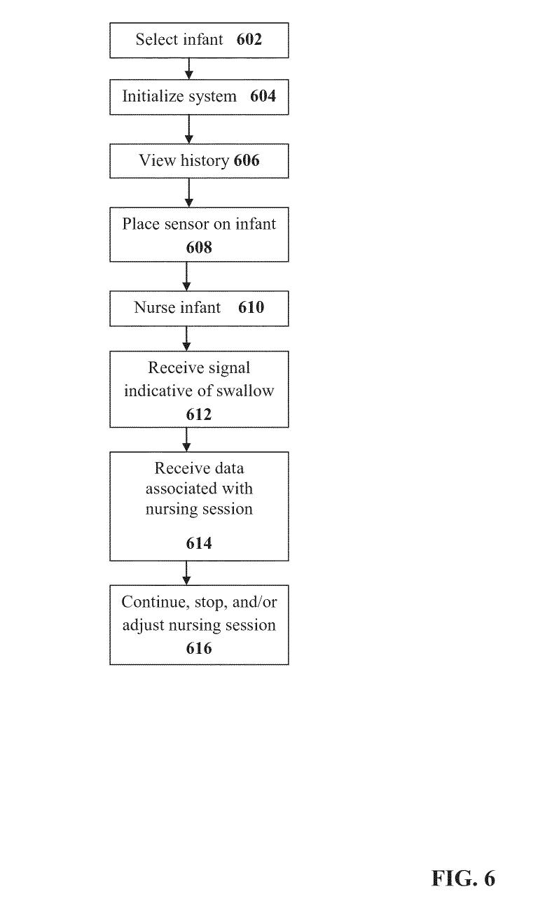

Reference is now made to FIG. 6, which is a flowchart of a method of nursing a baby by detecting swallowing events, in accordance with exemplary embodiments of the present invention. Optionally, the infant swallowing events are detected by the method described with reference to FIG. 1. Alternatively or additionally, the device of FIG. 3 and/or the stethoscope of FIG. 4 are used by the mother. Optionally, the stethoscope is connected to the mobile device of FIG. 5. Advantageously, the method provides the nursing mother with an indication of the swallowing event and/or augmentation of swallowing sounds.

Optionally, at 602, an infant is selected for automated detection of swallowing events, in accordance with exemplary embodiments of the present invention. The selection of the infant may be performed by the mother, or by recommendation from a healthcare provider.

Optionally, the infant is selected for augmentation of audible sounds. For example, the baby is over about 5 days old. Alternatively, the infant is selected for detection of inaudible sounds. Optionally, the swallowing sounds of the infant are of a volume that is too low for the mother to discern, for example, the baby is less than about 5 days old. The baby may be premature.

Optionally, at 604, the mother initializes the detection device, for example, as described with reference to initialization module 330 of FIG. 3. Optionally, demographic data is entered and/or updated. Optionally, the volume of milk per swallow is estimated according to the entered demographic data. Alternatively or additionally, the software is upgraded.

Optionally, at 606, the mother reviews the history of previous feeding sessions, for example, the total volume last consumed, total volume today, and/or number of swallows during the last feed. Reviewing the history may help the mother plan the upcoming feeding session.

At 608, the audio sensor is positioned on the infant. Optionally, the sensor is gently held against the neck of the feeding infant, for example, chestpiece 402 of FIG. 4. Alternatively or additionally, the sensor is taped and/or attached using an adhesive. Alternatively or additionally, the sensor is positioned near the infant without contact. The sensor may be positioned against and/or near other parts of the infant, for example, the temple, the upper part of the back, the upper part of the chest, or other locations that transmit swallowing sounds.

The sensor may be positioned with only one hand. Optionally, the hand may be removed after positioning leaving both hands free.

Optionally, at 610, the mother begins to nurse the infant.

Alternatively, the mother first positions the baby and ensures that the baby has latched on, and then places the sensor on the baby as in 608. The mother may begin to nurse before starting the automated swallowing event detection.

At 612, the mother receives a signal indicating that one or more swallowing events have been detected. For example, the mother may hear augmented sounds of the swallowing event, the mother may heard audio sounds and/or see images, as described herein in more detail.

Optionally, the device automatically detects the start of the feeding, for example, by detecting the first swallowing pattern. Alternatively or additionally, the mother presses a button to indicate that the feeding session has begun.

Optionally, the device monitors for detected errors, for example, low signal quality, and/or low battery. Optionally, the system advises the mother of any detected errors (e.g., by blinking light, and/or text message). Optionally, the device monitors the state of the detected errors. Optionally, the device continues the process of detecting the swallowing events after the mother corrects the source of the detected errors.

Optionally, at 614, the mother receives additional data associated with the nursing session. Data may be in real time, may be cumulative for the session, may be for the last detected event and/or may be for all previous sessions. For example, the number of swallowing events detected in this session, the total estimated volume of milk in this session (calculated by a linear model of multiplying the estimated volume of milk per swallow from box 604, by the number of detected swallowing events, and/or calculated by a non-linear model), quality signal measures, probability of the detected swallowing per event and/or average for the session, respiration data, and/or other data as described herein.

Optionally, at 616, the mother makes a decision associated with the feeding session. Optionally, the decision is assisted by the data of box 614. For example: the mother may decide to continue feeding if the infant did not consume enough milk, the mother may adjust the position of the baby if the event swallow rate is low, the mother may adjust the position of the sensor on the baby if the signal quality is low, the mother may move the baby to the other breast if about half of the milk has been consumed so far, and/or the mother may stop the feeding session if the infant consumed enough milk.

It is expected that during the life of a patent maturing from this application many relevant infant swallowing detection devices and/or methods will be developed and the scope of the term infant swallowing detection devices and/or methods is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of the present invention may be presented in a range format. It should be understood that the description in range format is merely for convenience and brevity and should not be construed as an inflexible limitation on the scope of the present invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the present invention, which are, for clarity, described in the context of separate embodiments, may also be provided in combination in a single embodiment. Conversely, various features of the present invention, which are, for brevity, described in the context of a single embodiment, may also be provided separately or in any suitable subcombination or as suitable in any other described embodiment of the present invention. Certain features described in the context of various embodiments are not to be considered essential features of those embodiments, unless the embodiment is inoperative without those elements.

1. A computerized method of detecting at least one infant swallowing event, the method comprising:
obtaining at least one signal segment of an audio recording of an infant during a breastfeeding session;
automatically detecting a first, a second and a third peak region in the at least one signal segment;
automatically calculating a first duration from the end of the first region to the start of the second region and a second duration from the end of the second region to the start of the third region;
automatically determining an infant swallowing event when the first duration falls within a first signature time range and the second duration falls within a second signature time range; and
outputting an audio signal indicative of the determined infant swallowing event to a user so that the user is aware of the infant swallowing event.

2. The computerized method of claim 1, wherein the first signature time range comprises about 50-300 ms and the second signature time range comprises about 300-1000 ms.

3. The computerized method of claim 1, further comprising providing initialization data associated with the infant to estimate an amount of milk per infant swallowing event, and estimating a total amount of milk swallowed per feeding session according the estimated milk per infant swallowing event and the number of detected infant swallowing events.

4. The computerized method of claim 1, wherein outputting comprises outputting an augmented filtered signal of the detected infant swallowing event.

5. The computerized method of claim 1, wherein the audio recording is recorded from an infant with swallowing sounds with a volume too low for the mother to discern.

6. The computerized method of claim 1, wherein the first duration corresponds to a pharyngeal stage of the infant swallowing event and the second duration corresponds to an esophageal stage of the infant swallowing event.

7. (canceled)

8. The computerized method of claim 1, wherein automatically detecting comprises automatically estimating a probability of the infant swallowing event.

9. The computerized method of claim 8, wherein the signal segment is scaled so that the signal segment is used to estimate the probability.

10. The computerized method of claim 1, further comprising detecting respiration sound patterns, associating the respiration sound patterns with the detected infant swallowing event, and increasing or decreasing the probability of the detected infant swallowing event according to the associations of the respiration patterns.

11. The computerized method of claim 1, further comprising monitoring the quality of the signal of the at least one signal segment and outputting a signal indicative of the quality so that a user may correct the signal quality.

12. A system for detecting at least one infant swallowing event during a breastfeeding session, the system comprising:
a sensor for detecting audio signals associated with at least one infant swallowing event, the sensor outputting at least one audio signal segment;
a processor adapted for executing a code for:
analyzing the at least one audio signal segment for an infant swallow pattern indicative of the at least one infant swallowing event, the processor comprising:
detecting a first, a second and a third peak regions in the at least one signal segment;
calculating a first duration from the end of the first peak region to the start of the second peak region and a second duration from the end of the second peak region to the start of the third peak region;
detecting that the at least one swallowing event has occurred when the first duration falls within a first signature time range and the second duration falls within a second signature time range; and
instructing a generation of a signal indicative of the swallowing event; and
an output unit for generating an output of one or both of a sound and a picture in response to the signal indicative of the swallowing event.

13. (canceled)

14. The system of claim 12, wherein the processor resides in a smartphone wherein the output unit comprises an audio headset and the sensor is attached to the headset.

15. (canceled)

16. The system of claim 12, further comprising a high pass filter to filter the at least one audio signal segment above 2.5 kHz to reduce or remove signal components due to one or both of respiration and voice.

17. The system of claim 12, further comprising one or both of a Gaussian filter and an exponential smoothing filter to filter the at least one audio signal segment so that multiple peaks will generate a relatively higher response.

18. The system of claim 12, further comprising a memory in electrical communication with the processor, the memory storing data of current and previous infant feeding sessions.

19. (canceled)

20. A stethoscope for listening to infant swallowing sounds comprising:
a contact element sized for positioning against the skin of an infant's head or neck;
a sensor for generating at least one electrical audio signal segment in response to received sound vibrations from the infant, the sensor is in communication with the contact element so that sound is transmitted from the infant through the contact element to the sensor;
an electrical transceiver for providing electrical communication external of the stethoscope, for transmitting the at least one electrical audio signal segment, and for receiving at least one signal indicative of an infant swallowing event; and
at least one speaker sized for positioning in proximity of or inside an ear canal of a user, the speakers being in electrical communication with the electrical transceiver so that the at least one signal indicative of the infant swallowing event is transmitted by the speakers to the ears of the user.

21. (canceled)

22. The stethoscope of claim 20, wherein the processor is further adapted for:
detecting a first, a second and a third peak regions in the at least one audio signal segment;
calculating a first duration from the end of the first peak region to the start of the second peak region and a second duration from the end of the second peak region to the start of the third peak region;
determining when the first duration falls within a first signature time range and the second duration falls within a second signature time range; and
generating the at least one signal indicative of the infant swallow.

23. The stethoscope of claim 20, wherein the speakers are detachable from the stethoscope.

24-25. (canceled)

26. The stethoscope of claim 20, wherein the electrical transceiver is an integrated Jack Plug for connecting to a mobile device.

27. The computerized method of claim 1, wherein the outputting comprises displaying an indication of said determined infant swallowing event on a display of a mobile device having a processor used for executing the computerized method.
